(19) United States
(12) Patent Application Publication
(10) Pub. No.: US 2022/0157449 A1
(43) Pub. Date: May 19, 2022

(54) AUTOMATED TRIAGE NURSE SYSTEM IN A VEHICLE

(71) Applicant: Ford Global Technologies, LLC, Dearborn, MI (US)

(72) Inventors: Stuart Salter, White Lake, MI (US); John Van Wiemeersch, Novi, MI (US); Peter Phung, Windsor (CA); Paul Kenneth Dellock, Northville, MI (US); Annette Huebner, Highland, MI (US); Kristin Hellman, Walled Lake, MI (US)

(73) Assignee: Ford Global Technologies, LLC, Dearborn, MI (US)

(21) Appl. No.: 17/098,296

(22) Filed: Nov. 13, 2020

(51) Int. Cl. G16H 40/67; G16H 80/00; G16H 10/20; G16H 50/20

(52) U.S. Cl. CPC G16H 40/67; G16H 50/20; G16H 10/20; G16H 80/00

(57) ABSTRACT

Example embodiments described in this disclosure are generally directed to an automated triage nurse system in a vehicle. In an example scenario, the system issues a set of questions to evaluate a severity of a medical emergency event associated with an occupant of the vehicle. The questions may be issued through an audio system in the vehicle and the medical emergency event may be caused by a vehicular accident or may be a medical trauma suffered by the occupant. The system provides medical advice to the occupant, if the occupant provides a response that indicates to the system that it is unnecessary to summon a first responder. The medical advice can be a first-aid instruction. On the other hand, the system may contact a first responder located in a medical facility if the occupant of the vehicle provides no response, or provides a response that indicates a serious medical problem.

### FIELD OF THE DISCLOSURE

This disclosure generally relates to operations associated with a vehicle and more particularly relates to an automated triage nurse system in a vehicle.

### BACKGROUND

Vehicle accidents can range from a minor accident in which none of the occupants of a vehicle are injured badly enough to warrant summoning an emergency responder, to a major accident in which one or more occupants of a vehicle are moderately to seriously injured and require attention from a medical professional. A medical professional, such as a paramedic riding in an ambulance, may take a certain amount of time to reach a site of a major accident. The amount of time taken can vary. In some cases, the ambulance may arrive at the accident site in a relatively short amount of time because a medical facility is located close to the accident site. In some other cases, no medical facility may be located close to the accident site and the ambulance may take a significant amount of time to reach the accident site. In either case, the medical professional has to spend a certain amount of time to diagnose injuries (by talking to an injured person or another occupant who may be able to assist the injured, for example), and to decide on what treatment to provide.

It is highly desirable to minimize the amount of time taken to perform such activities, particularly in cases where the injuries are life-threatening. It is also desirable for the medical professional to convey instructions to a person at the site of the accident (a passenger, for example, when the driver is injured) for providing first-aid to the injured, even before the medical professional reaches the site of the accident. In some cases, the medical professional may be unable to do so because of lack of adequate information on the nature of the injuries (the passenger may be disoriented, incoherent, or may lack communication skills).

### BRIEF DESCRIPTION OF THE DRAWINGS

A detailed description is set forth below with reference to the accompanying drawings. The use of the same reference numerals may indicate similar or identical items. Various embodiments may utilize elements and/or components other than those illustrated in the drawings, and some elements and/or components may not be present in various embodiments. Elements and/or components in the figures are not necessarily drawn to scale. Throughout this disclosure, depending on the context, singular and plural terminology may be used interchangeably.

FIG. 1 shows an example vehicle that includes an automated triage nurse system in accordance with the disclosure.

FIG. 2 shows some example components that may be included in the vehicle shown in FIG. 1.

FIG. 3 shows a first flowchart of an example method of operation of an automated triage nurse system 105 in accordance with the disclosure.

### DETAILED DESCRIPTION

### Overview

In terms of a general overview, certain embodiments described in this disclosure are directed to an automated triage nurse system in a vehicle. As is known, a triage nurse is typically a person who is a registered nurse working in an emergency room (ER) of a medical facility. The triage nurse is responsible for quickly evaluating a patient in order to determine appropriate treatment. In some cases, the patient may have been seriously injured in a vehicle collision or a gun-related incident, for example, and it is highly desirable that the triage nurse make an accurate evaluation with minimum delay so as to minimize the impact of the injury (prevent an amputation, for example) or to save the life of the patient.

In an example scenario in accordance with the disclosure, the automated triage nurse system that is provided in a vehicle issues a set of questions to evaluate a severity of a medical emergency event associated with an occupant of the vehicle. The set of questions may be transmitted through an audio system in the vehicle and the medical emergency event may be caused by a vehicular accident or may be a medical trauma suffered by the occupant of the vehicle (a heart attack or diabetic coma, for example). The automated triage nurse system provides medical advice to the occupant of the vehicle if the occupant of the vehicle provides a first type of response. The first type of response may indicate to the automated triage nurse system that it is unnecessary to summon a first responder. So, the automated triage nurse system offers medical advice such as, for example, a first-aid advice. On the other hand, the automated triage nurse system may contact a first responder located outside the vehicle (at a hospital, for example) if the occupant of the vehicle provides no response or provides a second type of response that indicates a serious medical problem.

### Illustrative Embodiments

The disclosure will be described more fully hereinafter with reference to the accompanying drawings, in which example embodiments of the disclosure are shown. This disclosure may, however, be embodied in many different forms and should not be construed as limited to the example embodiments set forth herein. It will be apparent to persons skilled in the relevant art that various changes in form and detail can be made to various embodiments without departing from the spirit and scope of the present disclosure. Thus, the breadth and scope of the present disclosure should not be limited by any of the above-described example embodiments but should be defined only in accordance with the following claims and their equivalents. The description below has been presented for the purposes of illustration and is not intended to be exhaustive or to be limited to the precise form disclosed. It should be understood that alternate implementations may be used in any combination desired to form additional hybrid implementations of the present disclosure. For example, any of the functionality described with respect to a particular device or component may be performed by another device or component. Furthermore, while specific device characteristics have been described, embodiments of the disclosure may relate to numerous other device characteristics. Further, although embodiments have been described in language specific to structural features and/or methodological acts, it is to be understood that the disclosure is not necessarily limited to the specific features or acts described. Rather, the specific features and acts are disclosed as illustrative forms of implementing the embodiments.

Certain words and phrases are used herein solely for convenience and such words and terms should be interpreted as referring to various objects and actions that are generally understood in various forms and equivalencies by persons of ordinary skill in the art. For example, the word “automated” as used herein refers to a computerized system that performs certain functions automatically without human intervention. The word “occupant” as used herein with reference to a vehicle is equally applicable to a driver of the vehicle or a passenger of the vehicle. The word “issuing” as used herein with reference to the automated triage nurse is intended to encompass many different forms of communication between a vehicle and an occupant of the vehicle. Such forms can include audible communications via an audio device in the vehicle (a loudspeaker, for example), a visual communication through a display screen (an onscreen image or video, for example), and a wireless signal transmitted to an audio device worn by the occupant of the vehicle (a phone earpiece, for example). Words such as “analyzing,” “evaluating,” and “processing” may be used interchangeably and generally refer to obtaining information from an animate or an inanimate object. The object may be, for example, an occupant of a vehicle, a photograph, or a video clip. Words such as “wireless” or “wirelessly” as used herein in the context of communication links are not intended to preclude other forms of communication links such as optical communication links, acoustic or ultrasonic communication links, and wired communication links that may be used alternatively. The words “sensor” and “detector” may be used interchangeably and generally refer to event sensing and data capture devices such as an accelerometer that detects force changes characteristic of a collision, a sensor that detects human body parameters (blood pressure, heart rate, body temperature etc.), a sensor that maps the position of occupants and contents such as a radar system, and a camera that detects facial expressions and provides information of a viewed area (status of occupants, extent of damage to structure, etc.). Furthermore, it should be understood that the word “example” as used herein is intended to be non-exclusionary and non-limiting in nature.

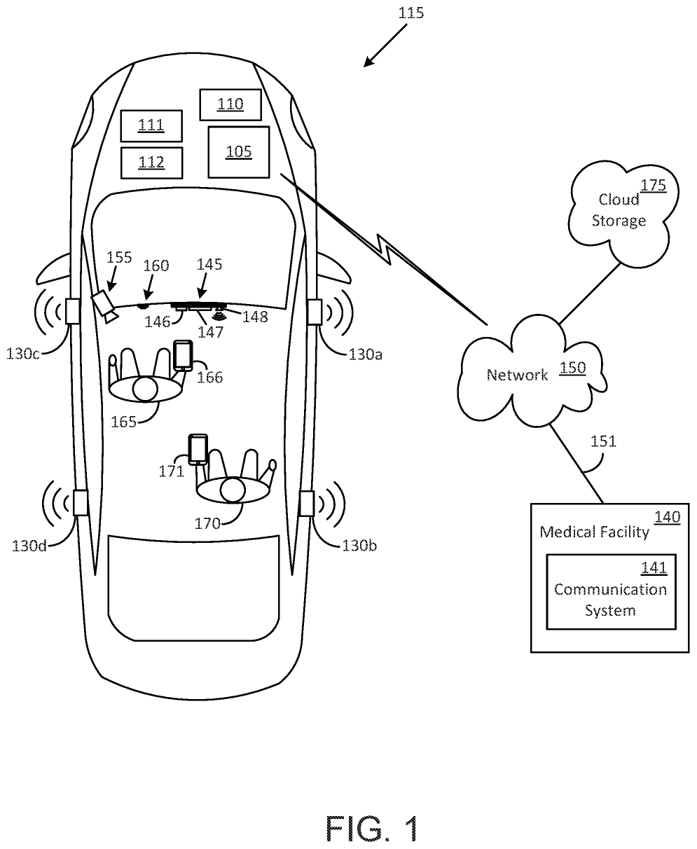

FIG. 1 shows an example vehicle 115 that includes an automated triage nurse system 105 in accordance with the disclosure. The vehicle 115 may be any of various types of vehicles such as a gasoline powered vehicle, an electric vehicle, a hybrid electric vehicle, or an autonomous vehicle, and can include other components such as a vehicle computer 110, a vehicle collision detection system 111, a vehicle communication system 112, and an infotainment system 145. In some examples described below, the vehicle 115 can include occupants such as a driver 165 and a passenger 170.

The vehicle computer 110 may perform various functions such as controlling engine operations (fuel injection, speed control, emissions control, braking, etc.), managing climate controls (air conditioning, heating etc.), and issuing warnings (check engine light, bulb failure, low tire pressure, vehicle in blind spot, etc.).

The vehicle collision detection system 111 performs functions such as detecting an impact associated with a collision or a spin-out or a roll-over associated with an unexpected driving hazard, and detecting an unexpected driving maneuver (braking, acceleration, etc.) associated with a driving hazard. The vehicle collision detection system 111 detects such events based on signals received from various sensors and detectors in the vehicle 115. In some implementations, the vehicle collision detection system 111 can be a part of the vehicle computer 110. The vehicle collision detection system 111 may incorporate an accelerometer sensor that transmits a signal to the vehicle computer 110 upon sensing events such as a sudden stop, a collision, a spin, a roll-over, or an unexpected deceleration. The sudden stop or deceleration may be caused when the driver 165 of the vehicle 115 steps on the brakes very hard during an emergency stop or when the vehicle 115 collides with an object such as another vehicle or a structure. The vehicle computer 110 may respond to the signal received from the vehicle collision detection system 111 by activating airbags or shutting-off fuel delivery in the vehicle 115.

The infotainment system 145 can be an integrated unit that includes various components such as a radio 146, a display screen 147, a speaker 148, and other components such as a USB access port, a Bluetooth® system, Ultra-Wide Band (UWB) system, and a global positioning satellite (GPS) navigation system. In an example implementation, the display screen 147 may include a graphical user interface (GUI) for use by an occupant of the vehicle 115. The GUI may be used for various purposes such as to enter commands for interacting with the GPS navigation system, to select a phone number for making a phone call, and to make a song selection. The GUI may also be used by the driver 165 and/or the passenger 170 to communicate with the automated triage nurse system 105 in accordance with the disclosure.

In some implementations, the automated triage nurse system 105 may be an independent unit. In some other implementations, some or all components of the automated triage nurse system 105 may be incorporated into the vehicle computer 110. The automated triage nurse system 105 may support wired and/or wireless communications with various devices inside and outside the vehicle 115. For example, the driver 165 may use the GUI on the infotainment system 145 that is coupled via wiring to the automated triage nurse system 105, or use a handheld device 166 (such as a cellular phone) to wirelessly communicate with the automated triage nurse system 105. The vehicle computer 110 can detect a damage to the GUI during an accident and automatically transfer various functionalities of the automated triage nurse system 105 to one or more personal devices in the vehicle 115, such as, for example, to a handheld device 171 of the passenger 170.

Some wireless technologies that can be used for the wireless communications can include, for example, Bluetooth®, Ultra-Wideband, Wi-Fi, Zigbee®, and near-field-communications (NFC). The automated triage nurse system 105 may communicate with other vehicles or other devices outside the vehicle 115 by using the vehicle communication system 112. The vehicle communication system 112 can include wireless communications devices such as, for example, a set of wireless communication nodes 130a, 130b, 130c, and 130d that are provided on the body of the vehicle 115, or a single wireless communication node that is mounted upon the roof of the vehicle 115.

In the example embodiment shown in FIG. 1, the automated triage nurse system 105 is communicatively coupled to a network 150 and a cloud storage element 175 via one or more of the wireless communication nodes 130a, 130b, 130c, and 130d. The network 150 may include any one, or a combination of networks, such as a local area network (LAN), a wide area network (WAN), a telephone network, a cellular network, a cable network, a wireless network, and/or private/public networks such as the Internet. For example, the network 150 may support communication technologies such as Bluetooth®, UWB, cellular, near-field communication (NFC), Wi-Fi, Wi-Fi direct, Li-Fi, audio-based communication, machine-to-machine communication, and/or man-to-machine communication. At least one portion of the network 150 supports communication between the wireless communication nodes 130a, 130b, 130c, and 130d on the vehicle 115 and a communication system 141 in a medical facility 140. The communication system 141 may be coupled to the network 150 via a communication link 151 that can be, for example, any of a wired link, a wireless link, an audio link, or an optical link.

The automated triage nurse system 105 is communicatively coupled to the communication system 141 located outside the vehicle 115 and various devices inside the vehicle 115 (such as the vehicle computer 110, the infotainment system 145, and handheld devices) for supporting various operations in accordance with the disclosure. The communicative coupling between the automated triage nurse system 105 and the vehicle computer 110 allows the automated triage nurse system 105 to receive various signals from the vehicle computer 110 such as, for example, a signal indicating that the vehicle computer 110 has deployed an airbag and/or a signal indicating that the driver 165 has hit the brakes very hard (even though an airbag has not been deployed). Such situations may cause a medical emergency event wherein an occupant of the vehicle 115 may suffer an injury. In some cases, the injury may be serious in nature such as, for example, a skull fracture, a concussion accompanied by a blackout, or a rib injury with damage to internal organs. Such injuries typically require immediate medical attention from a medical professional. In some other cases, the injury may be relatively minor in nature such as, for example, a cut, a bruise, or an abrasion. Such injuries may not require immediate medical treatment and may be taken care of through first-aid measures or by visiting a doctor at a later time.

In an example operation in accordance with the disclosure, the automated triage nurse system 105 receives a trigger that is associated with a medical emergency event. The trigger can be provided to the automated triage nurse system 105 in various forms such as, for example, in the form of a signal from the vehicle collision detection system 111, an input from an occupant of the vehicle 115, or input from an image capture device 155 in the vehicle 115. The signal from the vehicle collision detection system 111 may indicate to the automated triage nurse system 105 that an airbag has deployed in the vehicle 115. One example input from the occupant of the vehicle 115 may be in the form of a phone call to the automated triage nurse system 105 from the handheld device 166 of the driver 165 (or from the handheld device 171 of the passenger 170). Another example input from the occupant of the vehicle 115 may be a depression of a panic button 160 or an utterance of a unique triage nurse system activation password which may be detected by a device such as Siri® or Alexa®. Yet another example input from the occupant of the vehicle 115 may be via the GUI of the infotainment system 145.

Input provided by the image capture device 155 to the automated triage nurse system 105 may be obtained directly from the image capture device 155 or indirectly through the vehicle computer 110. Input obtained via the vehicle computer 110 can include optical and/or radar images of the cabin of the vehicle 115 and/or exterior of the vehicle 115 that have been processed by the vehicle computer 110, as well as other data (such as a speed of the vehicle 115 prior to an accident and a braking characteristic during the accident).

The image capture device 155 may be arranged to have a field of view that encompasses a cabin area of the vehicle 115 so as to capture images of the occupants of the vehicle 115. A video buffer may be provided in the image capture device 155 so as to store images or videos that are captured by the image capture device 155. The images may be analyzed by the automated triage nurse system 105 to evaluate a severity of a medical emergency event. Analyzing the images may include processing images in real-time after the vehicle 115 has experienced a collision, and may further include analyzing images obtained from the video buffer of events that occurred prior to the collision. Analysis of pre-collision and post-collision images may allow the automated triage nurse system 105 to evaluate a severity of the medical emergency event and the location status of occupants.

In one example scenario, the driver 165 may have been leaning forward prior to the time of collision and a skull injury caused by impact of the skull of the driver 165 upon the steering wheel may be more severe than if the head of the driver 165 had been well behind and above the steering wheel prior to the time of collision. In some implementations, images of areas outside the vehicle may be analyzed for evaluating a severity of the medical emergency event. The analyzing may be directed, for example, at determining a size of another vehicle that has collided with the vehicle 115, the number of vehicles involved in the collision, and a type of object that has come in contact with the vehicle 115 (wall, pillar, tree, etc.), or whether the occupants of the vehicle 115 are in the same position prior to the accident as they are after the accident.

Some types of medical emergencies experienced by an occupant of the vehicle 115 may be caused by factors unrelated to the vehicle 115. For example, an occupant of the vehicle 115 may suffer a seizure or a heart attack. The seizure or heart attack may, or may not, be followed by a vehicular accident. For example, the driver 165 may notice that the passenger 170 is undergoing a heart attack, and may safely pull over to a shoulder on the road. The automated triage nurse system 105 may be informed of the medical emergency in various ways. In one case, the driver 165 may press the panic button 160, which sends a trigger to the automated triage nurse system 105. In another case, the trigger may be a video clip received from the image capture device 155. The automated triage nurse system 105 may evaluate the video clip along with real-time video for evaluating the severity of the medical emergency event.

In some cases, an injury to an occupant of the vehicle 115 may be serious in nature such as, for example, a skull fracture, a concussion, a blackout, or a rib injury with damage to internal organs. Such injuries typically require immediate medical attention from a medical professional. Consequently, the automated triage nurse system 105 immediately contacts the closest medical facility and/or a first responder (a medical professional or an ambulance service, for example) without spending time interacting with the occupant of the vehicle 115. The closest medical facility (medical facility 140, for example) and/or the first responder may be contacted by the automated triage nurse system 105 in various ways such as via an automated phone call, an email, or a text message.

In one example scenario, the automated triage nurse system 105 may identify the medical facility and/or the first responder to be contacted by referring to data stored in a database of the automated triage nurse system 105. In another scenario, the automated triage nurse system 105 may contact the medical facility 140 by sending a communication to the communication system 141 in the medical facility 140. The communication may include details about the medical emergency event. A person monitoring the communication system 141 may respond to the communication by determining the appropriate medical personnel, medical equipment, and/or medical supplies to be used in view of the details of the medical emergency event that is provided by the automated triage nurse system 105. For example, a medical professional trained in dealing with skull fractures and head trauma may be dispatched along with suitable equipment if the medical emergency event involves a head injury. A different medical professional trained in dealing with heart attacks may be dispatched along with a defibrillator, for example, if the medical emergency event involves a heart attack, and so on.

The medical facility 140 may inform the automated triage nurse system 105 of the dispatch together with contact information of a medical professional traveling in an ambulance towards the accident site. The automated triage nurse system 105 may use the contact information to transmit to the medical professional, updates pertaining to the medical emergency event. The medical professional may use the automated triage nurse system 105 to provide audible instructions to an occupant of the vehicle 115 via the infotainment system 145, for example. The occupant of the vehicle 115 can be the passenger 170 and the audible instructions can include first-aid treatment procedures to perform upon an injured occupant of the vehicle 115.

In some other cases, the injury may be relatively minor in nature such as, for example, a cut, a bruise, or an abrasion. Such injuries may not require immediate medical treatment and may be taken care of through first-aid measures or by visiting a doctor at a later time. Consequently, the automated triage nurse system 105 may offer guidance and/or medical instructions via the infotainment system 145. The database of the automated triage nurse system 105 can include a number of medical instructions and the automated triage nurse system 105 may identify a subset of the medical instructions to be provided to the occupants of the vehicle based on the nature of the medical emergency event. Guidance provided by the automated triage nurse system 105 may further include information about the closest medical facility where the driver 165 can receive medical treatment.

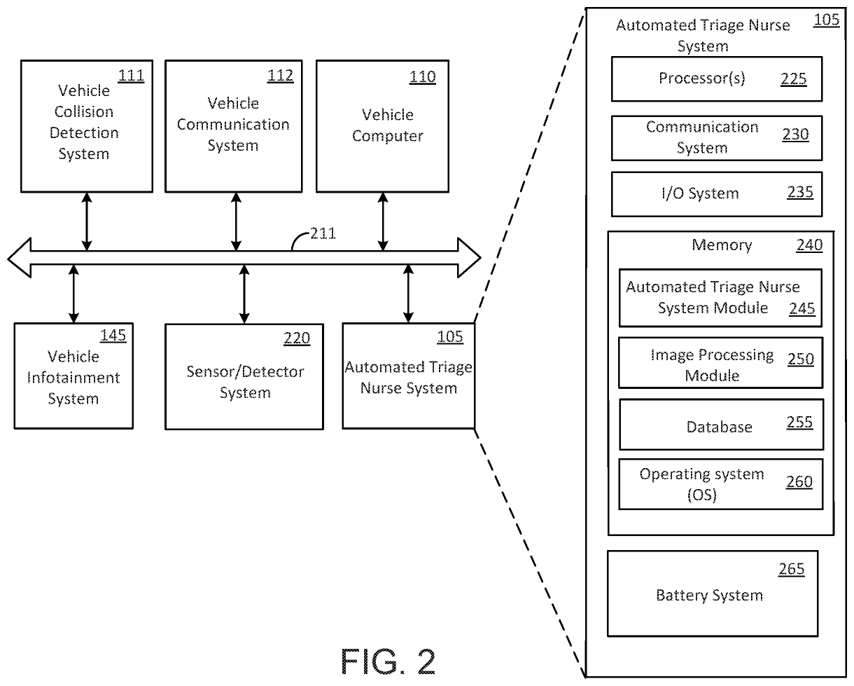

FIG. 2 shows some example components that may be included in the vehicle 115. The example components may include the vehicle collision detection system 111, the vehicle communication system 112, the vehicle computer 110, the infotainment system 145, a sensor/detector system 220, and the automated triage nurse system 105. The various components can be communicatively coupled to each other via one or more buses such as an example bus 211. The bus 211 may be implemented using various wired and/or wireless technologies. For example, the bus 211 can be a vehicle bus that uses a controller area network (CAN) bus protocol, a Media Oriented Systems Transport (MOST) bus protocol, and/or a CAN flexible data (CAN-FD) bus protocol. Some or all portions of the bus 211 may also be implemented using wireless technologies such as Bluetooth®, Bluetooth® Low Energy (BLE), Ultra-Wideband, Wi-Fi, Li-Fi, Zigbee®, or near-field-communications (NFC). For example, the bus 211 may include a Bluetooth® communication link that allows the vehicle collision detection system 111 and/or the vehicle computer 110 to wirelessly communicate with the automated triage nurse system 105.

The sensor/detector system 220 can include various types of sensors and detectors such as, for example, an accelerometer for detecting collisions and the image capture device 155 for capturing images.

The vehicle communication system 112 can include various wireless communication nodes such as the wireless communication nodes 130a, 130b, 130c, and 130d. In one example implementation, some or all of the wireless communication nodes can include a Bluetooth® low energy module (BLEM) and/or a Bluetooth® low energy antenna module (BLEAM).

The automated triage nurse system 105 may include a processor 225, a communication system 230, an input/output (I/O) system 235, a memory 240, and a battery system 265. The communication system 230 can include one or more wireless transceivers (BLEMs, for example) that allow the automated triage nurse system 105 to transmit and/or receive various types of data from the vehicle computer 110 via the vehicle communication system 112. The communication system 230 can also include hardware for communicatively coupling the automated triage nurse system 105 to the wireless communication nodes 130a, 130b, 130c, and 130d for carrying out machine-to-man communications such as with a first responder in an ambulance traveling towards an accident site, and machine-to-machine communications with the communications system 141 in the medical facility 140.

The I/O system 235 may include hardware that receives signals from various sensors of the sensor/detector system 220, such as, for example, a signal from the vehicle collision detection system 111 and/or images from the image capture device 155.

The memory 240, which is one example of a non-transitory computer-readable medium, may be used to store an operating system (OS) 260, a database 255, and various code modules such as an automated triage nurse system module 245 and an image processing module 250. The code modules are provided in the form of computer-executable instructions that can be executed by the processor 225 for performing various operations in accordance with the disclosure.

The automated triage nurse system module 245 may be executed by the processor 225 for performing various operations related to providing automated triage nurse services. For example, the automated triage nurse system module 245 may be executed by the processor 225 in response to a trigger received from the vehicle computer 110 via the I/O system 235. Responding to the trigger can include operations such as evaluating a severity of a medical emergency event and providing a first aid instruction to an injured occupant of the vehicle 115 when the injury is relatively minor in nature such as, for example, a cut, a bruise, or an abrasion. Such injuries may not require immediate medical treatment. When the injury is more severe, the automated triage nurse system module 245 may include computer-executable instructions that are executed by the processor 225 for automatically contacting a first responder and providing information to the first responder. The information may be conveyed to the first responder when the first responder is at the medical facility 140, is on his/her way to the vehicle 115 at an accident site, and/or is in the vehicle 115 at the accident site.

Evaluating the severity of a medical emergency event can include the use of artificial intelligence procedures. An example evaluation procedure can involve execution of the image processing module 250 for analyzing images obtained from the image capture device 155, for example. The images may be evaluated to obtain various types of information such as, for example, to determine an extent of injury to an occupant of the vehicle 115 and/or to evaluate damage to the vehicle 115 that may pose problems to a first responder. The extent of injury to the occupant may be evaluated based on features such as facial expressions, bleeding conditions, state of consciousness, responses to audible queries, and level of alertness. The damage to the vehicle 115 can include, for example, structural damage (occupant is trapped in the vehicle 115, for example), and hazardous conditions (gasoline leakage, toxic conditions, etc.).

In one example implementation, the database 255 may be used to store information that may be accessed by the processor 225 when executing the automated triage nurse system module 245. Some examples of such information may include medical remedies and treatment for minor injuries, first-aid instructions to stabilize an injured occupant of the vehicle 115, and contact information for contacting first responders and medical facilities. In some implementations, the database 255 can include personal data of one or more occupants of the vehicle 115, such as, for example, medical conditions (asthma, high blood pressure, diabetes, etc.), allergies (to antibiotics and certain drugs), emergency contacts, and medical history (surgeries, heart attacks, etc.). Information stored in the database 255 may be used by the processor 225 to perform various operations in accordance with the disclosure such as providing medical advice to an injured occupant, conveying information to a first responder, and contacting people upon occurrence of a medical emergency event.

In some implementations, where the database 255 lacks certain types of information, the processor 225 may obtain the information from a database in the medical facility 140 or from a cloud storage element 175. The database 255 may be used as a cache for storing various types of information retrieved from the cloud storage element 175. The cloud storage element 175 is typically configured to store a large amount of data in comparison to the smaller amount of storage available in the database 255. The database 255 may therefore be utilized in a strategic manner to store information that is more relevant to the occupant(s) of the vehicle 115 and is more quickly accessible. In an example implementation, the database 255 may be used as a cache for storing the latest information about treatment strategies and treatment-related information. Treatment-related information may include items such as addresses, accessibility, and capabilities of trauma centers located near any given area where the vehicle 115 is traveling at any given time and/or has traveled in the past (for example, a regularly traveled route to an office or a school). The cached information in the database 255 may be particularly useful when the communication system 230 is unable to communicate with the cloud storage element 175 for various reasons such as, for example, due to lack of cellular capacity in a rural area.

The battery system 265 provides power for operating the various components of the automated triage nurse system 105 under normal conditions as well as when the vehicle 115 has been involved in an accident. In some implementations, the battery system 265 may be an integral part of a battery system provided in the vehicle 115. In some other implementations, the battery system 265 may be an independent unit that is configured to remain operational and provide power to the automated triage nurse system 105 even when the battery system of the vehicle is damaged or disabled during an accident.

It must be understood that even though the various components in FIG. 2 are shown as discrete functional blocks, some of these components, or some parts of these components, may be combined together in some implementations in accordance with the disclosure. For example, in one example implementation, the processor 225 of the automated triage nurse system 105 may be a part of the vehicle computer 110 or the vehicle collision detection system 111.

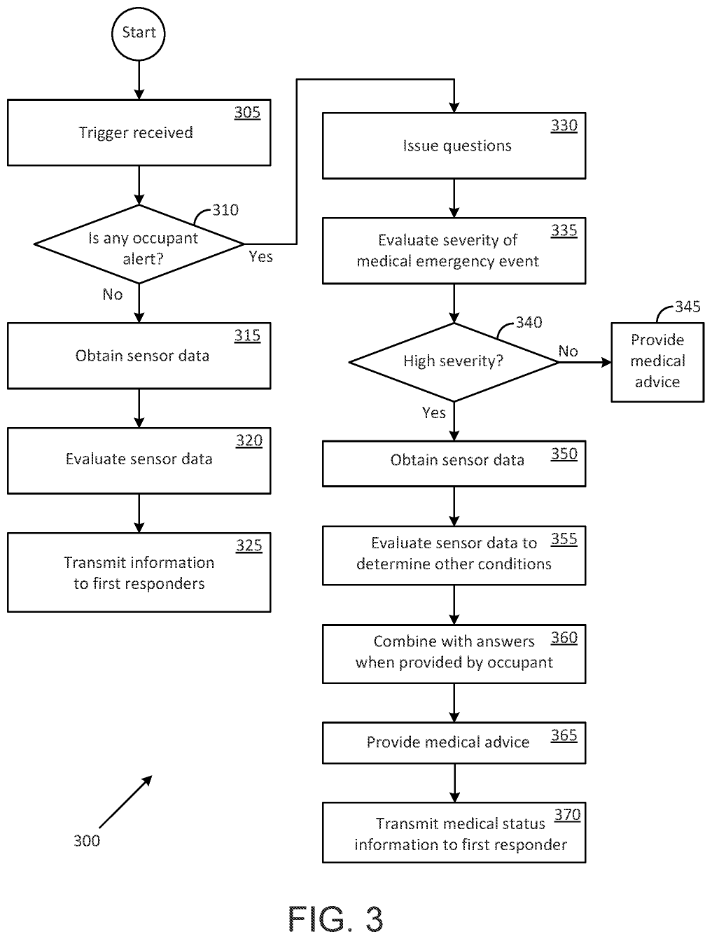

FIG. 3 shows a flowchart 300 of an example method of operation of the automated triage nurse system 105 in accordance with the disclosure. The flowchart 300 illustrates a sequence of operations that can be implemented in hardware, software, or a combination thereof. In the context of software, the operations represent computer-executable instructions stored on one or more non-transitory computer-readable media such as the memory 240, that, when executed by one or more processors such as the processor 225, perform the recited operations. Generally, computer-executable instructions include routines, programs, objects, components, data structures, and the like that perform particular functions or implement particular abstract data types. The order in which the operations are described is not intended to be construed as a limitation, and any number of the described operations may be carried out in a different order, omitted, combined in any order, and/or carried out in parallel. The description below may make reference to certain components and objects shown in FIG. 1 and FIG. 2, but it should be understood that this is done for purposes of explaining certain aspects of the disclosure and that the description is equally applicable to many other embodiments.

At block 305, the automated triage nurse system 105 receives a trigger that is associated with a medical emergency event. The trigger can be provided to the automated triage nurse system 105 in various forms such as, for example, in the form of a signal from the vehicle collision detection system 111, an input from an occupant of the vehicle 115, or input from a component of the sensor/detector system 220. The signal from the vehicle collision detection system 111 may indicate to the automated triage nurse system 105 that an airbag has deployed in the vehicle 115. One example input from the occupant of the vehicle 115 may be in the form of a phone call to the automated triage nurse system 105 from the handheld device 166 of the driver 165 (or from the handheld device 171 of the passenger 170). Another example input from the occupant of the vehicle 115 may be a depression of a panic button 160. Yet another example input from the occupant of the vehicle 115 may be via the GUI of the infotainment system 145.

At block 310, the automated triage nurse system 105 makes a determination about an alertness state of one or more occupants of the vehicle 115. In one example scenario, the automated triage nurse system 105 makes the determination by analyzing real time video received from the image capture device 155. The video may show the driver 165 slumped unconscious on the steering wheel and the passenger 170 attempting to help the driver 165 by talking to the driver 165 and/or extending a hand to lift the driver 165 off the steering wheel. In another situation, the video may show all the occupants of the vehicle 115 have been rendered unconscious and are not moving.

At block 315, the automated triage nurse system 105 may obtain sensor data from the sensor/detector system 220 in the vehicle 115 and/or from the vehicle computer 110 if all the occupants of the vehicle 115 are unconscious or are unable to communicate with the automated triage nurse system 105.

At block 320, the data obtained from the sensor/detector system 220 may be evaluated. For example, the automated triage nurse system 105 may obtain data from an accelerometer sensor and/or from the vehicle computer 110 to determine a severity of a collision. As another example, the automated triage nurse system 105 may process images received from the image capture device 155 to identify various parameters such as name, address, and health status of one or more occupants of the vehicle 115. This process may be carried out by using reference images and data stored in the database 255 of the automated triage nurse system 105 or in the cloud storage element 175. As yet another example, the automated triage nurse system 105 may process images received from the image capture device 155 to perform a medical evaluation to determine the nature and extent of injuries suffered by the occupants. This process may be carried out by using reference medical data stored in the database 255 of the automated triage nurse system 105 or in the cloud storage element 175. The medical evaluation may also be carried out by processing data from sensors such as a blood pressure sensor, a heart rate sensor, and other body sensors that may be mounted on various objects in the vehicle 115 such as, for example, the steering wheel, the driver's seat, and passenger seats.

At block 325, the information collected and evaluated by the automated triage nurse system 105 may be transmitted to various entities including first responders. For example, the automated triage nurse system 105 may transmit information about the accident to a fire department and a police department (911, for example). Information about a medical condition of the occupants of the vehicle 115 may be transmitted by the automated triage nurse system 105 to a medical professional. The medical professional may be a first responder traveling in an ambulance on the way to the medical emergency location (accident site, for example) or a person in the medical facility 140. Such information not only allows the medical professional to take certain pre-emptive actions, such as bringing appropriate medicines, drugs, or equipment to the medical emergency location, but also saves time for providing treatment to the injured occupants of the vehicle 115. Saving time is highly desirable in most medical emergencies and can be critical in some cases such as when an occupant has suffered a head injury or is experiencing a heart attack.

Back at block 310, the automated triage nurse system 105 may make a determination that one or more occupants of the vehicle 115 are alert and can provide input to the automated triage nurse system 105. For example, an analysis of real time video received from the image capture device 155 video may indicate to the automated triage nurse system 105 that the driver 165 is slumped unconscious on the steering wheel and the passenger 170 attempting to help the driver 165 by talking to the driver 165 and/or extending a hand to lift the driver 165 off the steering wheel.

At block 330, the automated triage nurse system 105 may issue a set of questions and/or requests to the occupant of the vehicle 115. The questions and/or requests may be provided in audible form through the speaker 148 of the infotainment system 145. In an example implementation, the automated triage nurse system 105 may default to issuing the questions and/or requests in a language preferred by the occupant of the vehicle 115. However, the automated triage nurse system 105 may switch to a different language when the occupant issues a command to do so. The command can be an oral command or an entry through a keyboard of the automated triage nurse system 105. In some cases where the occupant has configured the automated triage nurse system 105 to issue questions and requests in a preferred language (such as, for example, Spanish), and the vehicle 115 is being operated in a country or region where the local language is other than Spanish, the automated triage nurse system 105 may offer to switch to the local language or to a default language (English, for example).

At block 335, answers provided by an occupant of the vehicle 115 may be analyzed by the automated triage nurse system 105 to evaluate a severity of the medical emergency event (vehicle accident, heart attack, etc.). The analysis may be based on data stored in the database 255 of the automated triage nurse system 105 or in the cloud storage element 175. The analysis may also be based on artificial intelligence applied for medical diagnosis.

A few example questions and requests that may be issued by the automated triage nurse system 105 are: “Are you hurt?”, “Where are your hurting?”, “Please rate your pain level between 1 and 10”, “Can you move?”, “Do you have shortness of breath?”, “Do you have chest pain?”, “Are you numb in any part of your body?”, “Are you experiencing tingling in any part of your body?”, “Do you have a headache?”, “Please provide a brief summary of your medical history”, “Have you been admitted to a hospital in the past?”, “Have you had surgery before?”, “Are you allergic to antibiotics?”, and “Are you allergic to any medications?”

At block 340, the automated triage nurse system 105 makes a determination regarding the severity of the medical emergency event. In one implementation, the determination may be made exclusively based on the answers provided by the occupant(s) of the vehicle 115. In another implementation, the determination may be made on the basis of a combination of answers provided by the occupant(s) of the vehicle 115 and data received from various sensors in the vehicle 115.

In one example scenario, the automated triage nurse system 105 may determine that the severity of the medical emergency event is low, based on receiving a first type of response from an occupant of the vehicle 115. For example, the occupant may describe a medical condition that is relatively minor in nature such as, for example, an injury involving a small cut, a bruise, or an abrasion. Such injuries may not require immediate medical treatment and may be taken care of through first-aid measures or by visiting a doctor at a later time. Consequently, at block 345, the automated triage nurse system 105 offers guidance and/or medical instructions to the occupant.

In another example scenario, the automated triage nurse system 105 may determine that the severity of the medical emergency event is high, based on receiving no response from an occupant or receiving a second type of response that indicates a serious medical condition. For example, the occupant may appear to be alert (eyes open, moving around etc.) but may be unable to answer one or more questions due to having suffered a concussion or due to a blackout while answering a question issued by the automated triage nurse system 105.

At block 350, the automated triage nurse system 105 may obtain sensor data from various sensors and detectors in the vehicle 115 and/or from the vehicle computer 110.

At block 355, the sensor data may be evaluated for obtaining information about the vehicle 115 and conditions such as a severity of a collision and structural damage to the vehicle 115. The sensor data may also be evaluated for obtaining information about conditions that may be relevant to providing medical assistance to an injured occupant (for example, occupant is trapped behind steering wheel, blood flowing out of the neck of the occupant, and incoherent speech). In some cases, the evaluation may be based on artificial intelligence procedures.

At block 360, the information obtained by evaluating data obtained from the sensors and detectors are combined with information provided by the occupant(s) of the vehicle 115 when the patient is able to provide answers to questions such as described above.

At block 365, the automated triage nurse system 105 offers guidance and/or medical instructions to the occupant having high severity injuries. For example, the occupant may be unable to actively participate in any conversation with the automated triage nurse system 105 and/or render medical aid to himself/herself and to other occupants of the vehicle. In such a situation, the automated triage nurse system 105 may offer palliative care such as, for example, in the form of providing encouragement and/or repeatedly urging the injured occupant to stay awake.

At block 370, the combined information may be transmitted to various entities. For example, the automated triage nurse system 105 may transmit information about the accident to entities such as a fire department or a police department (911, for example). Information about a medical condition of the occupants of the vehicle 115 may be transmitted by the automated triage nurse system 105 to a medical professional. The medical professional may be a first responder traveling in an ambulance on the way to the medical emergency location (accident site, for example) or a person in the medical facility 140. In an example implementation, one or more people associated with the vehicle such as, for example, an owner of the vehicle 115, the driver 165 of the vehicle 115, or the passenger 170 of the vehicle 115 may provide a waiver that legally permits the automated triage nurse system 105 to transmit medical information to various entities such as a medical professional. The waiver may be obtained at suitable times such as, for example, at a time of registration of the automated triage nurse system 105 with a service entity such as, for example, an insurance company or a rental car agency. The waiver may be tailored to conform to Health Insurance Portability and Accountability Act (HIPPA) rules and regulations.

In some cases, the information provided by the automated triage nurse system 105 to the medical professional or other entity, may be insufficient, ambiguous, or inaccurate. The medical professional can query the automated triage nurse system 105 to obtain answers or to obtain clarification. The queries may be transmitted to the automated triage nurse system 105 via a computer system in an ambulance or in the medical facility 140, and may involve man-to-machine interaction incorporating technologies such as voice recognition and voice synthesis.

Some vehicles are equipped to automatically unlock all doors of the vehicle when the vehicle has been rendered motionless after being involved in certain types of accidents. The doors are unlocked so at allow any good Samaritan to provide aid to the injured occupant(s) of the vehicle 115. In such cases, the automated triage nurse system 105 may interactively communicate with the good Samaritan in order to assist the good Samaritan render aid.

In the above disclosure, reference has been made to the accompanying drawings, which form a part hereof, which illustrate specific implementations in which the present disclosure may be practiced. It is understood that other implementations may be utilized, and structural changes may be made without departing from the scope of the present disclosure. References in the specification to “one embodiment,” “an embodiment,” “an example embodiment,” “an example embodiment,” etc., indicate that the embodiment described may include a particular feature, structure, or characteristic, but every embodiment may not necessarily include the particular feature, structure, or characteristic. Moreover, such phrases are not necessarily referring to the same embodiment. Further, when a particular feature, structure, or characteristic is described in connection with an embodiment, one skilled in the art will recognize such feature, structure, or characteristic in connection with other embodiments whether or not explicitly described.

Implementations of the systems, apparatuses, devices, and methods disclosed herein may comprise or utilize one or more devices that include hardware, such as, for example, one or more processors and system memory, as discussed herein. An implementation of the devices, systems, and methods disclosed herein may communicate over a computer network. A “network” is defined as one or more data links that enable the transport of electronic data between computer systems and/or modules and/or other electronic devices. When information is transferred or provided over a network or another communications connection (either hardwired, wireless, or any combination of hardwired or wireless) to a computer, the computer properly views the connection as a transmission medium. Transmission media can include a network and/or data links, which can be used to carry desired program code means in the form of computer-executable instructions or data structures and which can be accessed by a general purpose or special purpose computer. Combinations of the above should also be included within the scope of non-transitory computer-readable media.

Computer-executable instructions comprise, for example, instructions and data which, when executed at a processor, cause the processor to perform a certain function or group of functions. The computer-executable instructions may be, for example, binaries, intermediate format instructions such as assembly language, or even source code. Although the subject matter has been described in language specific to structural features and/or methodological acts, it is to be understood that the subject matter defined in the appended claims is not necessarily limited to the described features or acts described above. Rather, the described features and acts are disclosed as example forms of implementing the claims.

A memory device such as the memory 240, can include any one memory element or a combination of volatile memory elements (e.g., random access memory (RAM, such as DRAM, SRAM, SDRAM, etc.)) and non-volatile memory elements (e.g., ROM, hard drive, tape, CDROM, etc.). Moreover, the memory device may incorporate electronic, magnetic, optical, and/or other types of storage media. In the context of this document, a “non-transitory computer-readable medium” can be, for example but not limited to, an electronic, magnetic, optical, electromagnetic, infrared, or semiconductor system, apparatus, or device. More specific examples (a non-exhaustive list) of the computer-readable medium would include the following: a portable computer diskette (magnetic), a random-access memory (RAM) (electronic), a read-only memory (ROM) (electronic), an erasable programmable read-only memory (EPROM, EEPROM, or Flash memory) (electronic), and a portable compact disc read-only memory (CD ROM) (optical). Note that the computer-readable medium could even be paper or another suitable medium upon which the program is printed, since the program can be electronically captured, for instance, via optical scanning of the paper or other medium, then compiled, interpreted or otherwise processed in a suitable manner if necessary, and then stored in a computer memory.

Those skilled in the art will appreciate that the present disclosure may be practiced in network computing environments with many types of computer system configurations, including in-dash vehicle computers, personal computers, desktop computers, laptop computers, message processors, handheld devices, multi-processor systems, microprocessor-based or programmable consumer electronics, network PCs, minicomputers, mainframe computers, mobile telephones, PDAs, tablets, pagers, routers, switches, various storage devices, and the like. The disclosure may also be practiced in distributed system environments where local and remote computer systems, which are linked (either by hardwired data links, wireless data links, or by any combination of hardwired and wireless data links) through a network, both perform tasks. In a distributed system environment, program modules may be located in both the local and remote memory storage devices.

Further, where appropriate, the functions described herein can be performed in one or more of hardware, software, firmware, digital components, or analog components. For example, one or more application specific integrated circuits (ASICs) can be programmed to carry out one or more of the systems and procedures described herein. Certain terms are used throughout the description, and claims refer to particular system components. As one skilled in the art will appreciate, components may be referred to by different names. This document does not intend to distinguish between components that differ in name, but not function.

It should be noted that the sensor embodiments discussed above may comprise computer hardware, software, firmware, or any combination thereof to perform at least a portion of their functions. For example, a sensor may include computer code configured to be executed in one or more processors and may include hardware logic/electrical circuitry controlled by the computer code. These example devices are provided herein for purposes of illustration and are not intended to be limiting. Embodiments of the present disclosure may be implemented in further types of devices, as would be known to persons skilled in the relevant art(s).

At least some embodiments of the present disclosure have been directed to computer program products comprising such logic (e.g., in the form of software) stored on any computer-usable medium. Such software, when executed in one or more data processing devices, causes a device to operate as described herein.

While various embodiments of the present disclosure have been described above, it should be understood that they have been presented by way of example only, and not limitation. It will be apparent to persons skilled in the relevant art that various changes in form and detail can be made therein without departing from the spirit and scope of the present disclosure. Thus, the breadth and scope of the present disclosure should not be limited by any of the above-described example embodiments but should be defined only in accordance with the following claims and their equivalents. The foregoing description has been presented for the purposes of illustration and description. It is not intended to be exhaustive or to limit the present disclosure to the precise form disclosed. Many modifications and variations are possible in light of the above teaching. Further, it should be noted that any or all of the aforementioned alternate implementations may be used in any combination desired to form additional hybrid implementations of the present disclosure. For example, any of the functionality described with respect to a particular device or component may be performed by another device or component. Further, while specific device characteristics have been described, embodiments of the disclosure may relate to numerous other device characteristics. Further, although embodiments have been described in language specific to structural features and/or methodological acts, it is to be understood that the disclosure is not necessarily limited to the specific features or acts described. Rather, the specific features and acts are disclosed as illustrative forms of implementing the embodiments. Conditional language, such as, among others, “can,” “could,” “might,” or “may,” unless specifically stated otherwise, or otherwise understood within the context as used, is generally intended to convey that certain embodiments could include, while other embodiments may not include, certain features, elements, and/or steps. Thus, such conditional language is not generally intended to imply that features, elements, and/or steps are in any way required for one or more embodiments.

That which is claimed is:

1. A method comprising:
receiving, by an automated triage nurse system in a vehicle, a trigger that is associated with a medical emergency event;
evaluating, by the automated triage nurse system, a severity of the medical emergency event; and
contacting, by the automated triage nurse system, a first responder, based on evaluating the severity of the medical emergency event.

2. The method of claim 1, wherein the trigger is provided to the automated triage nurse system by one of a vehicle collision detection system, an occupant of the vehicle, or an image capture device in the vehicle.

3. The method of claim 1, wherein the first responder is located outside the vehicle.

4. The method of claim 1, wherein evaluating the severity of the medical emergency event comprises the automated triage nurse system issuing a set of questions to an occupant of the vehicle.

5. The method of claim 4, wherein the first responder is contacted by the automated triage nurse system subject to one of receiving no response from the occupant of the vehicle or receiving a request for medical assistance from the occupant of the vehicle.

6. The method of claim 4, further comprising:
issuing, by the automated triage nurse system, a medical advice to the occupant of the vehicle based on answers provided by the occupant of the vehicle.

7. The method of claim 6, wherein the automated triage nurse system generates the medical advice by applying an artificial intelligence procedure.

8. A method comprising:
issuing, by an automated triage nurse system, a set of questions to evaluate a severity of a medical emergency event associated with an occupant of a vehicle;
providing, by the automated triage nurse system, a medical advice to the occupant of the vehicle based on receiving a first type of response from the occupant of the vehicle; and
contacting, by the automated triage nurse system, a first responder outside the vehicle, based on one of receiving a second type of response from the occupant of the vehicle or receiving no response from the occupant of the vehicle.

9. The method of claim 8, further comprising:
receiving, by the automated triage nurse system in the vehicle, a trigger indicative of the medical emergency event; and
providing, by the automated triage nurse system, to the first responder, information pertaining to the medical emergency event.

10. The method of claim 9, wherein the first responder is one of traveling towards the vehicle, is in the vehicle or is located in a medical facility.

11. The method of claim 8, wherein the medical emergency event is caused by a vehicular accident or a medical trauma suffered by the occupant of the vehicle.

12. The method of claim 8, wherein evaluating the severity of the medical emergency event comprises the automated triage nurse system accessing a database.

13. The method of claim 12, wherein the database is located in one of the vehicle or in a cloud storage element outside the vehicle.

14. The method of claim 8, wherein the set of questions are medical diagnosis questions that are transmitted by the automated triage nurse system through an audio system in the vehicle.

15. An automated triage nurse system in a vehicle, the automated triage nurse system comprising:
a memory that stores computer-executable instructions; and
a processor configured to access the memory and execute the computer-executable instructions to at least:
issue a set of questions to evaluate a severity of a medical emergency event associated with an occupant of the vehicle;
provide a medical advice to the occupant of the vehicle based on receiving a first type of response from the occupant of the vehicle; and
contact a first responder outside the vehicle, based on one of receiving a second type of response from the occupant of the vehicle or receiving no response from the occupant of the vehicle.

16. The automated triage nurse system of claim 15, wherein the set of questions are medical diagnosis questions that are transmitted by the automated triage nurse system through an audio system in the vehicle.

17. The automated triage nurse system of claim 15, wherein the processor is configured to access the memory and execute additional computer-executable instructions to:
receive a trigger indicative of the medical emergency event; and
provide, to the first responder, information pertaining to the medical emergency event.

18. The automated triage nurse system of claim 17, wherein the trigger is provided to the automated triage nurse system by one of a vehicle collision detection system, an occupant of the vehicle, or an image capture device in the vehicle.

19. The automated triage nurse system of claim 15, wherein the processor is configured to access the memory and execute additional computer-executable instructions to:
generate the medical advice by applying an artificial intelligence procedure.

20. The automated triage nurse system of claim 15, wherein the first type of response from the occupant of the vehicle indicates a first level of severity and the second type of response from the occupant of the vehicle indicates a second level of severity that is greater than the first level of severity.
